(19) United States
(12) Patent Application Publication
(10) Pub. No.: US 2009/0007294 A1
(43) Pub. Date: Jan. 1, 2009

(54) Genetic Engineering of Male Sterility in Plants

(76) Inventor: Henry Daniell, Winter Park, FL (US)

Correspondence Address: Beusse Wolter Sanks Mora & Maire, 390 N. ORANGE AVENUE, SUITE 2500, ORLANDO, FL, 32801, US

(21) Appl. No.: 11/575,090

(22) PCT Filed: Sep. 13, 2005
(86) PCT No.: PCT/US05/32402

§ 371 Date: Jul. 18, 2007

Related U.S. Application Data
(60) Provisional application No. 60/606,285, filed on Sep. 1, 2004.

(51) Int. Cl. A01H 1/00; A01H 5/00; C12N 15/00; C12N 15/82

(52) U.S. Cl. 800/278; 435/320.1; 800/293; 800/298; 800/312; 800/314; 800/317.2; 800/317.3; 800/317.4; 800/320; 800/320.1; 800/320.2; 800/320.3

(57) ABSTRACT

Disclosed herein are methods of achieving male sterility in plants. Specifically exemplified herein is the transformation of the plastid genome with a vector expressing the phaA gene. Expression of the phaA gene in plastids results in plants that do not exhibit pleiotropic effects with the exception of male sterility. Also disclosed are stably transformed plants and cells, as well as example vectors for expressing the phaA gene in plastids.

### CROSS-REFERENCE TO RELATED APPLICATIONS

This application claims benefit of the Sep. 13, 2004, filing date of U.S. provisional patent application No. 60/609,285.

### STATEMENT REGARDING FEDERALLY SPONSORED RESEARCH

The work of this invention was supported in part by NIH grant no R01 GM 63879 and U.S.D.A. grant no. 0.3611-21000-017-00D to Henry Daniell.

### BACKGROUND

Male-sterility-inducing cytoplasms are known for over 100 years. Bateson and Gairdner (1) reported that male sterility in flax was inherited from the female parent. Chittenden and Pellow (2) observed that male sterility in flax was due to an interaction between the cytoplasm and nucleus. Jones and Clarke (3) established that male sterility in onion is conditioned by the interaction of the male-sterile (S) cytoplasm with the homozygous recessive genotype at a single male-fertility restoration locus in the nucleus. The authors also described the technique used today to exploit cytoplasmic-genic male sterility (CMS) for the production of hybrid seed. CMS inbred lines have been widely used for hybrid production of many crops. The first application of organelle biotechnology was the role played by cytoplasmic male sterility in hybrid seed production, a major contribution towards the “Green Revolution”. The use of cytoplasmic male sterility in hybrid seed production has been recently reviewed by Havey (4).

The use of CMS for hybrid seed production received a “black eye” after the epidemic of Bipolaris maydis on T-cytoplasmic maize (5). This epidemic is often cited as a classic example of genetic vulnerability of our major crop plants. In addition to Southern corn blight (CMS-T), cold susceptibility (CMS Ogura) and Sorghum Ergot infection in the unfertilized stigma have been reported (6, 7). But these disease linkages were successfully broken by somatic cell genetics and conventional plant breeding. Hybrids of other crop plants may be produced using nuclear male sterility. A natural source of nuclear male sterility was identified in leek (8). Engineered sources of nuclear male sterility have been developed in model systems (9, 10, 11). A problem with these nuclear transformants is that they segregate for male fertility or sterility and must be over planted and rogued by hand or sprayed with herbicides to remove male-fertile plants. Male-sterility systems have been created by different mechanisms, most of these affect tapetum and pollen development (12, 13, 14). Unfortunately, additional severe phenotypic alterations that were due to interference with general metabolism and development had precluded its use in agriculture (15, 16, 17).

Havey (4) documents the worldwide use of CMS to produce competitive hybrid cultivars. Major investments of time and resources are required to backcross a male-sterility-inducing cytoplasm into elite lines. These generations of backcrossing could be avoided by transformation of an organellar genome of the elite male-fertile inbred to produce female inbred lines for hybrid seed production. Because the male-fertile parental and male-sterile transformed lines would be developed from the same inbred, they should be highly uniform and possess the same nuclear genotype, excluding mutations and residual heterozygosity (4). Therefore, the male-fertile parental line becomes the maintainer line to seed-propagate the newly transformed male-sterile line (4). A few generations of seed increases would produce a CMS-maintainer pair for hybrid seed production. An additional advantage of organellar transformation would be the diversification of CMS sources used in commercial hybrid-seed production. Transformation of the chloroplast genome would allow breeders to introduce different male-sterility-inducing factors into superior inbred lines. Introduction of a male-sterility inducing transgene into one of the organellar genomes of a higher plant would be a major breakthrough in the production of male-sterile inbred lines (4). This technique would be of great potential importance in the production of hybrid crops by avoiding generations of backcrossing, an approach especially advantageous for crop plants with longer generation times (4). Moreover, transgenes that are engineered into our annual crops could be introgressed into wild crops, persist in the environment and have negative ecological consequences may be necessary to engineer a male sterility system that is 100% effective (18).

PHB synthesis takes place by the consecutive metabolic action of β-ketothiolase (phaA gene), acetoacetyl-CoA reductase (phaB) and PHB synthase (phaC). Poirier et al., (19) reported the expression of PHB in plants for the first time by expressing the phaB and phaC genes in the cytosol via nuclear transformation; taking advantage of available cytosolic acetoacetyl-CoA. This approach yielded very low levels of PHB; but severe pleiotropic effects were observed in the transgenic plants. In an attempt to increase the PHB yield in plants, Nawrath et al (20) introduced the phbA, phbB and phbC genes in individual nuclear Arabidopsis transgenic lines and reconstructed the entire pathway, targeting all enzymes to the plastids. This approach resulted in PHB expression up to 14% leaf dry weight, and no pleiotropic effects. This suggested that the depletion of metabolites from essential metabolic pathways in the cytoplasm might have caused the pleiotropic effects, and that by targeting the enzymes to chloroplast, which is a compartment with high flux through acetyl-CoA, the adverse effects were overcome (20). When expression of optimized gene constructs, PHB yield increased up to 40% leaf dry weight, but this was accompanied by severe growth reduction and chlorosis (21), indicating that targeting the PHB pathway to the chloroplast can result in pleiotropic effects, at higher concentrations of polymer synthesis (21). Lossl et al. (22) reported the expression of PHB in tobacco by expressing phaA, phaB and phaC via plastid transformation. The expression of PHB resulted in severe growth reduction and authors concluded that in tobacco significant levels of PHB could only be achieved if a sufficient pool of acetyl-CoA precursor is generated (22). Additionally, they observed that when the transgenic plants were grown autotrophycally, PHB levels significantly decreased which overcame the stunted phenotype, but male sterility was still observed. It was not known whether the polymer or other metabolic factors were responsible for the male sterile phenotype (22).

In an attempt to address the role of phaA expression in the pleiotropic effects observed in transgenic plants expressing PHB, Bohmert et. al., expressed the phbA gene constitutively and under inducible promoters via the nuclear genome (23). Constitutive expression of the phbA gene led to a significant decrease in transformation efficiency, inhibiting the recovery of transgenic lines and prevented analysis of plants expressing the β-ketothiolase gene (23). Such toxic effect exerted by phbA expression was speculated to be the result of PHB biosynthesis intermediates or its derivatives, the depletion of the acetyl-CoA pool, or of interaction of the β-ketothiolase with other proteins or substrates (23).

### SUMMARY OF THE INVENTION

The subject invention is directed to engineered male sterile phenotype in plants. The subject invention is based on the inventors' discovery that transformation of the β-ketothiolase (phaA gene) into the chloroplast genome, and its expression, enables the regeneration of transgenic plants that have overcome pleiotropic effects such as stunted phenotype and chlorosis observed during polyhydroxybutyrate expression (21, 23) but maintained complete (100%) male sterility.

### BRIEF DESCRIPTION OF THE DRAWINGS

FIG. 1. Molecular characterization of transgenic lines. A, Schematic representation of the transformed chloroplast genome and the pLDR-5′UTR-phaA cassette. Annealing sites for the primer pairs and expected sizes of PCR products are shown. BamHI restriction sites, DNA fragment produced after restriction digestion and the phaA probe, used in Southern-blot analyses are shown. B, the map shows the wild-type chloroplast genome, restriction digestion sites used for Southern-blot analysis, expected fragment size after digestion and probing with the flanking sequence.

FIG. 2 A, PCR analysis of wild type and putative transformants. Top panel, transgenic line 4B; bottom panel is transgenic line 4A. A, 1: Untransformed wild type; 2: 3P-3M (1.65 kb); 3: 4P-4M (1.65 kb); 4: 5P-2M (3.56 kb); 5: 5P-3′phaA (2.0 kb); 6: 5P-phaA internal (1.5 kb); 7: positive control (pLD-5′UTR-phbA plasmid DNA) 5P-phbA internal (1.5 kb); M: marker. B and D, Southern-blot analysis of T₀ and T₁ generation transgenic lines, respectively, with the chloroplast flanking sequence probe; 10 kb fragment shows integration of transgenes, 7.1 kb fragment shows wild type fragment. C and E, transgenic T₀ and T₁ plants, respectively, and wild type plant probed with the phaA probe. 10 kb fragment observed in transgenic lines but not in the wild type. F and G, northern-blot analysis using the phaA and aadA probes, respectively. Mononocistron (a: 5′UTR-phaA; 1,384 nt) and polycistrons (b: aadA-phaA, 2,255 nt; c: from native 16S Prrn, 4,723 nt) containing the phaA gene are observed in the transgenic lines when the phaA is used. Only polycistrons are observed with the aadA probe. In B to G: 1, untransformed plant; 2 to 4, chloroplast transgenic lines 4A, 4B, and 4C, respectively; B, blank lane.

FIG. 3 β-ketothiolase characterization in transgenic lines. A, coomassie-stained gel shows abundant β-ketothiolase expression in transgenic lines; 15 μg total plant protein was loaded per lane (a: D1 protein; b: β-ketothiolase, 40.8 kDa; c: RBCL). M: marker; 1-3: transgenic lines 4A, 4B, 4C, respectively; 4: 4A T₁ generation; 5: wild type; 6: bacterial purified β-ketothiolase. B, western-blot analysis: 10 μg total plant protein from transgenic lines and wild type was loaded per lane; anti-β-ketothiolase antibody was used. 40.8 kDa monomers (a), dimer (b), trimer (c) and tetramer (d) can be observed in transgenic lines. 1: untransformed plant; 2-4: transgenic lines 4A, 4B, 4C, respectively; 5-6: 4A and 4B T₁ generation, respectively.

FIG. 4. Characterization of male sterility, growth and development. A-C, Flowers from transgenic lines; note the absence of fruit capsules and fallen flowers. D and E, wild type tobacco flowers and fruit capsules. F and G, comparison of stamens and stigma. Note shorter stamens in transgenic lines (F) compared to untransformed (G). H and I, comparison of mature anthers. Note abundant pollen in untransformed plant (I) and the lack of pollen in transformed anther (H). J, transgenic fruit capsule with seeds developed after pollination of transgenic stigma with untransformed pollen. K, germination and growth of T₁ seedlings on MS medium with 500 μg/ml spectinomycin. wt: Untransformed; 4A: T₁ transgenic line 4A; 4B: T₁ transgenic line 4B, obtained after pollination with WT pollen.

FIG. 5 Comparison of growth and development. A, Untransformed (WT) and T₀ generation transgenic (T) line (4A) grown for two months in soil. B, Untransformed plant (WT) and T₁ generation independent transgenic lines 4A, 4B, 4C and 4D 1.5 month after germination.

FIG. 6 SEM pictures of pollen grains in anthers of wild type (A-C) and transgenic (D-F) plants at different magnifications. A, D: ×500; B, E: ×1000; C: ×3500; F: ×3000.

FIG. 7. β-Ketothiolase Expression in Anthers. A, pigmentation of anthers during flower development in transgenic plants, Stage 1 of flower development is shown. B, northern-blot analysis of flower and anthers. Monocistrons and polycistron are observed. 3 μg of total plant RNA was loaded per lane and the phaA probe was used. M: marker; 1: transgenic flower; 2-3: transgenic anthers; 4: wild type flower; 5: transgenic leaf; 6: wild type leaf. C, β-ketothiolase expression in transgenic flower and anther detected by western-blot analysis, lanes 2 and 3, respectively. RNA and protein samples used per lane were the product of the combined extraction from flowers or anthers from stages 1 and 3.

FIG. 8 Analysis of anther development. Bright-field photographs of untransformed (wt) and transgenic anthers at different developmental stages (S). Anthers at the designated stages were fixed, embedded with paraffin, and sliced into 5 and 10 μm transverse sections. The fixed sections were stained with toluidine blue and visualized under the light microscope at a magnifications of ×100. C, connective tissue; E, epidermis; En, endothecium; MMC, microspore mother cells; Msp, microspores; PS, pollen sac; S, stornium; T, tapetum.

FIG. 9 Reversibility of male fertility after 10 days under continuous illumination. A, transgenic flower after 9 days in continuous light. A, note normal length of the anther filaments and pollen grains. B, fully developed fruit capsules containing seeds from transgenic lines. C, abundant seeds from transgenic fruit capsule. D, seedlings produced via the reversibility to male fertility. Transgenic seeds germinated in MS medium supplied with 500 μm spectinomycin. E, bleached wild type tobacco seedlings.

FIG. 10 shows the nucleic acid sequence of the phaA gene.

### DETAILED DESCRIPTION

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

### DEFINITIONS

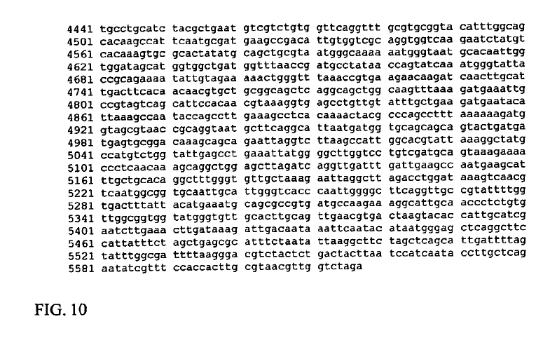

In the context of the present application, a polynucleotide sequence is “homologous” with the polynucleotide sequence according to the invention (Acinetobacter sp. β-ketothiolase gene, also referred to as phaA gene, accession no: L37761, see also FIG. 10) if at least 70%, preferably at least 80%, most preferably at least 90% of its base composition and base sequence corresponds to the sequence according to the invention; wherein corresponds refers to the percent identity of the nucleic acid sequences; or percent identity of the amino acid sequences that the nucleic acid sequences encode to the amino acid sequences described herein, as determined by algorithms commonly employed by those skilled in this art; or those sequences that are characterized by their ability to hybridize to a sequence according to the invention (as described below). In a specific embodiment, percent identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned, nucleic acid sequences. In another specific embodiment, suitable nucleic acid fragments (isolated polynucleotides of the present invention) encode polypeptides that are at least about 70% identical, preferably at least about 80% identical to the amino acid sequences described herein. Preferred nucleic acid fragments encode amino acid sequences that are at least about 85% identical to the amino acid sequences described herein. More preferred nucleic acid fragments encode amino acid sequences that are at least about 90% identical to the amino acid sequences described herein. Most preferred are nucleic acid fragments that encode amino acid sequences that are at least about 95% identical to the amino acid sequences described herein.

According to the invention, a “homologous protein” is to be understood to comprise proteins which contain an amino acid sequence at least 70% of which, preferably at least 80% of which, most preferably at least 90% of which, corresponds to the amino acid sequence disclosed in (Gish and States, 1993; L37761); wherein corresponds is to be understood to mean that the corresponding amino acids are either identical or are mutually homologous amino acids. The expression “homologous amino acids” denotes those which have corresponding properties, particularly with regard to their charge, hydrophobic character, steric properties, etc. Thus, the protein may be from 70% up to less than 100% identical to Acinetobacter sp. β-ketothiolase (accession no: L37761).

Sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The present invention also relates to plant cells or plants transformed with polynucleotides which contain the complete gene with the polynucleotide sequence corresponding to the phaA gene or fragments thereof, and which can be obtained by screening by means of the hybridization of a corresponding gene bank with a probe which contains the sequence of said polynucleotide molecule or a fragment thereof, and isolation of the DNA sequence.

Polynucleotide sequences according to the invention are suitable as hybridization probes for RNA, cDNA and DNA, in order to isolate those cDNAs or genes which exhibit a high degree of similarity to the sequence of the Acinetobacter sp. β-ketothiolase gene.

Polynucleotide sequences according to the invention are also suitable as primers for polymerase chain reaction (PCR) for the production of DNA which encodes an enzyme having aspartate decarboxylase activity.

Oligonucleotides such as these, which serve as probes or primers, can contain more than 30, preferably up to 30, more preferably up to 20, most preferably at least 15 successive nucleotides. Oligonucleotides with a length of at least 40 or 50 nucleotides are also suitable.

The term “isolated” means separated from its natural environment.

The term “polynucleotide” refers in general to polyribonucleotides and polydeoxyribonucleotides, and can denote an unmodified RNA or DNA or a modified RNA or DNA.

The term “polypeptides” is to be understood to mean peptides or proteins which contain two or more amino acids which are bound via peptide bonds.

The polypeptides for use in accord with the teachings herein include polypeptides corresponding to Acinetobacter sp. β-ketothiolase, and also includes those, at least 70% of which, preferably at least 80% of which, are homologous with the polypeptide corresponding to β-ketothiolase, and most preferably those which exhibit a homology of least 90% to 95% with the polypeptide corresponding to Acinetobacter sp. β-ketothiolase and which have enzymatic activity. Thus, the polypeptides may have a homology of from 70% to up to 100% with respect to Acinetobacter sp. β-ketothiolase.

The invention also relates to transforming plant cells and plants with polynucleotide sequences which result from phaA gene by degeneration of the genetic code. In the same manner, the invention further relates to DNA sequences which hybridize with phaA gene or with parts of phaA gene. Moreover, one skilled in the art is also aware of conservative amino acid replacements such as the replacement of glycine by alanine or of aspartic acid by glutamic acid in proteins as “sense mutations” which do not result in any fundamental change in the activity of the protein, i.e. which are functionally neutral. It is also known that changes at the N- and/or C-terminus of a protein do not substantially impair the function thereof, and may even stabilize the function.

In the same manner, the present invention also relates to employing DNA sequences which hybridize with phaA gene or with parts of phaA gene, or the complements thereof. Finally, the present invention relates to DNA sequences which are produced by polymerase chain reaction (PCR) using oligonucleotide primers which result from phaA gene. Oligonucleotides of this type typically have a length of at least 15 nucleotides.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% form amide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tm can be approximated from the equation of Meinkoth and Wahl, Anal. Biochem., 138:267-284 (1984): Tm=81.5° C.+16.6(log M)+0.41(% GC)−0.61(% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Current Protocols in Molecular Biology, Chapter 2, Ausubel, et al., Eds., Greene Publishing and Wiley-Interscience, New York (2000).

Thus, with the foregoing information, the skilled artisan can identify and isolate polynucleotides which are substantially similar to β-ketothiolase genes utilized in accord with the teachings herein.

In one embodiment, it may be advantageous for propagating the polynucleotide to carry it in a bacterial or fungal strain with the appropriate vector suitable for the cell type. Common methods of propagating polynucleotides and producing proteins in these cell types are known in the art and are described, for example, in Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989).

In another preferred embodiment, the polynucleotide sequence introduced into plastids according to the teachings herein comprises a phaA gene, polynucleotides which are complementary to phaA gene, and polynucleotides which are at least 70%, 80% or 90% identical to phaA gene.

### EXAMPLE 1

### Chloroplast Vector Construction

Plasmid DNA from Acinetobacter sp coding for the phaA gene (pJKD 1425) was provided by Metabolix (Cambridge, Mass.). Isolation and amplification of the phaA gene from the native plasmid was performed by polymerase chain reaction (PCR) with the utilization of phaA specific 5′ and 3′ flanking DNA primers. All primers were designed using the QUICKPRI program of the DNASTAR software. The PCR product was cloned into the vector pCR2.1-5′UTRpsbA, which contained the functional psbA gene promoter and 5′ regulatory sequence, by directional cloning after NdeI and NotI restriction digestion of the PCR product and vector. The phaA gene and the 5′UTRpsbA region were sequenced and subsequently cloned into the chloroplast transformation vector pLD-ctv, by directional insertion using appropriate restriction enzymes.

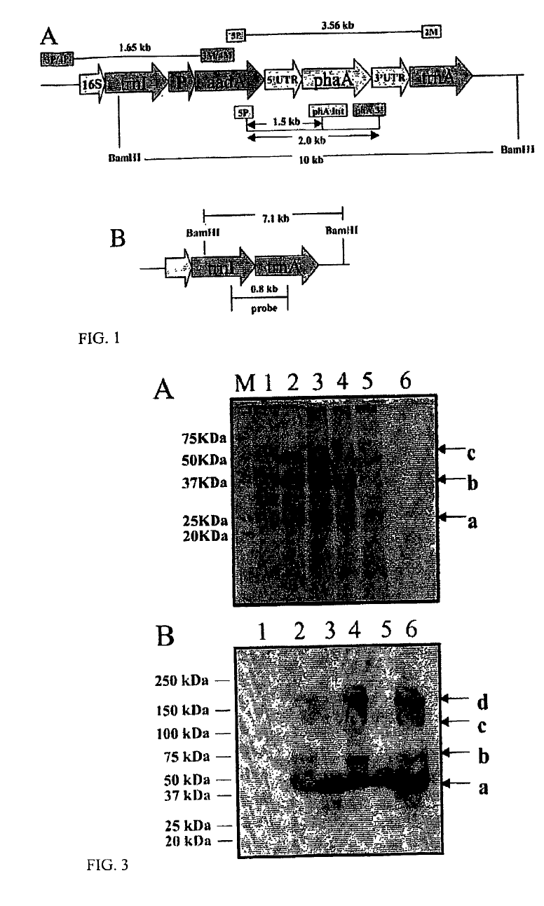

The Acinetobacter sp (accession no: L37761, sequence available via NCBI website www.ncbi.nlm.nih.gov) gene, phaA (1179 bp) coding for β-ketothiolase was amplified by PCR and cloned into the chloroplast transformation vector (pLD-ctv) to finally produce the pLDR-5′UTR-phaA vector (FIG. 1A). The vector contains chloroplast DNA sequences (flanking sequences), which allow site-specific integration by homologous recombination into the inverted repeat region of the chloroplast genome in between the trnI (tRNA Ile) and trnA (TRNA Ala) genes (27). This specific targeting mechanism allows high efficiency integration of the transgene construct containing the aadA (aminoglicoside 3′-adenylyltransferase) gene, which confers spectinomycin resistance and the phaA genes. The transcription of the aadA and phaA gene is driven by the constitutive action of the chloroplast 16S ribosomal RNA gene promoter (Prrn), which is located upstream of the aadA gene and should produce dicistrons (aadA-phaA). Additionally, the psbA gene promoter and 5′ regulatory sequence (5′ untranslated region; 5′ UTR), which is known to enhance translation of foreign genes in the light (28, 29, 30, 31), was used upstream of the phaA gene and should produce phaA monocistron. At the 3′ end of the gene construct is the 3′ psbA untranslated region (3′UTR), which is known to be involved in mRNA abundance and stability in chloroplast (32).

### EXAMPLE 2

### Chloroplast Transformation and Selection of Transgenic Plants

The delivery of the pLDR-5′UTR-phaA-3′UTR vector to the chloroplast by particle bombardment and the subsequent selection process of the transgenic tobacco (Nicotiana tabacum var bombarded using the biolistic device PDS-1000/He (Bio-Rad, Hercules, Calif.). After bombardment, leaves were placed on Regeneration Medium of Plants (RMOP), supplied with 500 μg mL⁻¹ spectinomycin for two rounds of selection on plates, and subsequently moved to jars on Murashige Skoog medium containing 500 μg mL⁻¹ spectinomycin. Finally, homoplasmic plants were transferred to high nutrient soil and grown in a controlled growth chamber at a temperature of 26° C. in a 16-h/8-h light/dark photoperiod.

### EXAMPLE 3

### Confirmation of Chloroplast Integration by PCR

Isolated total plant DNA from untransformed and transgenic plants using the DNeasy Plant Mini Kit (Qiagen, Valencia, Calif.) was used as the template for PCR reactions. The PCR primer pairs 3P-3M and 4P4M were used to confirm the integration of the gene cassette into the chloroplast, essentially as described previously (34). Primer pair 5P-2M, 5P-phaA internal and 5P-3′phaA were used to confirm the presence of the phaA gene. PCR analysis was performed using the Gene Amp PCR System 2400 (Perkin Elmer, Chicago).

### EXAMPLE 4

### Southern-Blot Analysis

The total plant DNA was obtained from T0 and T1 transgenic plants as well as from untransformed tobacco plants using the DNeasy Plant Mini Kit (Qiagen, Valencia, Calif.) and protocol. Southern blot analyses were performed essentially as described previously (34). Two μg of plant DNA was restriction digested with BamHI and resolved on a 0.8% (w/v) agarose gel at 50 V for 2 h. The gel was soaked in 0.25 N HCl for 15 min and was then rinsed two times with water. The gel was then soaked in transfer buffer (0.4 N NaOH and 1 M NaCl) for 20 min and the denatured DNA was transferred overnight to a nitrocellulose membrane by capillarity. The next day the membrane was rinsed twice in 2×SSC (0.3 M NaCl and 0.03 M sodium citrate), dried on Whatman paper, and then cross-linked in the GS GeneLiker (Bio-Rad, Hercules, Calif.) at setting C3 (150 njouls). The flanking sequence probe was obtained by BglII/BamHI restriction digestion of plasmid pUC-ct, which contains the chloroplast flanking sequence (trnI and trnA genes). The phaA probe was obtained by NdeI/NotI restriction digestion of plasmid pCR2.1-5′UTR-phaA. Probes were radio labeled with ³²P dCTP by using Ready Mix and Quant G-50 micro columns for purification (Amersham, Arlington Heights, Ill.). Prehybridization and hybridization were performed using the Quick-Hyb solution (Stratagene, La Jolla, Calif.). The membrane was washed twice for 15 min at room temperature in 2×SSC with 0.1% (w/v) SDS, followed by two additional washes at 60° C. (to increase the stringency) for 15 min with 0.1×SSC with 0.1% (w/v) SDS. Radiolabeled blots were exposed to x-ray films and then developed in the Mini-Medical Series x-ray film processor (AFP Imaging, Elmsford, N.Y.).

### EXAMPLE 5

### Northern-Blot Analysis

Total plant RNA from untransformed and chloroplast transgenic plants, was isolated by using the RNeasy Mini Kit (Qiagen, Valencia, Calif.) and protocol. Northern blot analyses were performed essentially as described previously (49). Total RNA (2.5 μg) per plant sample was resolved in a 1.2% (w/v) agarose/formaldehyde gel. The phaA probe generation, labeling reaction, prehybridization/hybridization, membrane washing steps, and autoradiography were performed essentially as explained above in the Southern-Blot section.

### EXAMPLE 6

### Western-Blot Analysis

Protein samples were obtained from 100 mg of leaf material from wild type and transgenic lines by grinding the tissue to a fine powder in liquid nitrogen, subsequent homogenization in 200 μl plant protein extraction buffer (100 mM NaCl, 10 mM EDTA, 200 mM Tris-HCl, 0.05% (w/v) Tween-20, 0.1% (w/v) SDS, 14 mM βP-mercaptoethanol (BME), 400 mM sucrose and 2 mM phenylmethylsulfonyl fluoride) and a centrifugation step at 15.7×g for 1 minute to remove solids. Protein concentrations were determined by Bradford assay (Bio-Rad Protein Assay) with bovine serum albumin as the protein standard. Proteins were resolved by electrophoresis in a 12% (v/v) SDS-PAGE and then transferred to a nitrocellulose membrane (Bio-Rad, Hercules, Calif.). The membrane was blocked for 1 hr with PTM buffer: 1×PBS (phosphate buffer solution), 0.05% (v/v) Tween-20 and 3% (w/v) non-fat dry milk. The membrane was probed for 2 hrs with rabbit anti-p-ketothiolase antibody (Metabolix, Cambridge, Mass.) in a dilution of 1:1,000, then rinsed with water twice and probed with alkaline phosphatase-conjugated secondary antibody (goat anti-rabbit, Sigma) for 1.5 hrs in a 1:20,000 dilution. Finally, the membrane was washed 3 times for 10 minutes with PT buffer (1×PBS, 0.05% (v/v) Tween-20) and one time with 1×PBS, followed by incubation in Lumi-phos WB (Pierce, Rockford, Ill.) reagent for the alkaline phosphatase reaction. Film exposure took place for 3 minutes.

### EXAMPLE 7

### β-Ketothiolase Activity Assay

Protein samples were obtained by grinding 1 g of leaf tissue to a fine powder in liquid nitrogen, followed by the addition of 2 ml ice cold β-ketothiolase extraction buffer (100 mM Tris-HCl pH 8.1, 50 mM MgCl₂, 5 mM BME) and homogenization. The homogenates were centrifuged for 10 minutes at 4° C. at 5,000 g, and the supernatant was passed through PD-10 columns (Amersham, Arlington Heights, Ill.) containing Sephadex G-25 M for desalting, and elution was optimized for the recovery of proteins of size range 25 to 60 kDa. Protein concentration was determined by a Bradford assay. β-ketothiolase activity was measured spectrophotometrically at 304 nm in the thiolysis direction (breaking down acetoacetyl-CoA to acetyl-CoA) by monitoring the disappearance of acetoacetyl-CoA for 60 seconds, which in the presence of Mg ion forms a magnesium enolate with absorbance at 304 nm; this protocol is an adaptation of the protocol by Senior and Davis (55). The reaction took place in a total volume of 1 ml containing 62.4 mM Tris-HCl pH 8.1, 50 mM MgCl₂, 62.5 μM CoA, 62.5 μM Acetoacetyl-CoA (substrate is dissolved in 50 mM phosphate buffer pH 4.7), 10 μl of plant extract (β-ketothiolase sample), and bringing the volume with distilled water to 1 ml. The plant extract containing the β-ketothiolase was added at the end immediately before the sample reading. In this assay, the enzyme specific activity is given in units per mg of total plant protein and 1 unit is defined as the degradation of 1 μmol/min of acetoacetyl-CoA under standard reaction conditions.

### EXAMPLE 8

### Scanning Electron Microscopy

Scanning electron microscopy (SEM) was performed at the AMPAC facility at the University of Central Florida. Anthers and pollen samples were gold coated on a Sputter Coater (Emitech, Houston, Tex.) with a gold film thickness of 150 Amstroms. SEM pictures were produced using the scanning electron microscope model JSM-6400F (JEOL, Peabody, Mass.), and the x-ray energy dispersive spectrometer (Edax, Mahwah, N.Y.) at an acceleration voltage of 6 kV.

### EXAMPLE 9

### Histological Analysis of Anthers

Anthers at relevant developmental stages were dissected from flower buds and fixed in 3% (v/v) glutaraldehyde in phosphate buffer for 12 hours at room temperature, applying a continuous vacuum for the first 3 hrs of incubation and degassing (by bringing the vacuum up and down slowly) for 10 min at 1 hr, 2 hr and 3 hr. The fixed anthers were dehydrated in an ethanol series (5%, 10% to 80% in increments of 10, 95% and 100%) for 30 min per gradient treatment. Samples were kept overnight in fresh 100% ethanol and were washed twice the next day for 1 hr in 100% ethanol. Samples were then treated with a gradient (25% to 100%) of Citro Solv clearing (Fisher, Pittsburgh, Pa.) reagent for 30 min per gradient treatment, and finally embedded in Paraplast Plus (Fisher, Pittsburgh, Pa.). Tissue embedding was performed in molten paraffin for 3 days, changing the molten paraffin every 8 to 12 hrs. Paraffin treated tissue was finally embedded into paraffin blocks by using the Leica EG 1160 paraffin embedding station (Leica, Solms). A metal blade microtome, model HM 315 (MICROM, Walldorf) was used for tissue embedded sectioning. Finally, tissue sections were put onto Superfrost/Plus microscope slides, followed by a rehydration step and tissue staining with 0.05% (w/v) toluidine blue. Tissue slides were observed under the Olympus BX60F5 light microscope and Olymppus U-CMAD-2 camera (Olympus, Melville, N.Y.). Flower developmental stages were characterized following the procedure described by Koltunow et al (56).

### EXAMPLE 10

### Reversibility of Male Fertility

Two independent transgenic plants were moved to a separate growth chamber after the first indication of flower bud formation and were kept away from any contact with wild type and other transgenic lines; the flowers were covered with thin transparent plastic bags to inhibit any possibility of cross pollination. Bags were only removed to take pictures. Transgenic plants were kept under continuous illumination for 10 days with a photon flux density of 11,250 μEm-2 supplied throughout this period. The number of flowers developed was counted daily throughout these ten days, while newly formed flowers, senescent flowers, and fallen flowers were recorded. The development of fruit capsules and seeds were also counted. After the 10 days, a 16 hrs light/8 hrs dark photoperiod was reestablished, while the plants were kept from contact with any other plant for 20 days to allow maturation of the fruit capsules and to harvest seeds produced during continuous illumination.

### EXAMPLE 11

### Transformation, Selection and Characterization of Chloroplast Transgenic Plants

Chloroplast transgenic plants were obtained through particle bombardment following the method described previously (33, 34, 35). More than 10 positive independent transgenic lines were obtained. Several independent transgenic lines were characterized, confirming that independent chloroplast transgenic lines show little variation in foreign gene expression (26). PCR based analysis with the primer pairs, 3P and 3M and 4P4M were used to test the integration of the transgene construct into the chloroplast genome (36). The 3P and 4P primers land on the native chloroplast genome, upstream of the gene cassette, and the 3M and 4M primers land on the aadA gene, which is located within the gene cassette (FIG. 1A). If site-specific integration had occurred, a PCR product of 1.65 kb should be obtained; this product was detected in transgenic lines (FIG. 2A lanes 2, 3). Untransformed plants as well as mutant plants, which had undergone spontaneous mutation of the 16S rRNA gene and acquired resistance to spectinomycin, did not show any PCR product, indicating that these plants are negative for integration (FIG. 2A lane 1). The integration of the aadA gene, as well as the phaA was further confirmed by the use of primer pairs 5P-2M, 5P-3′phaA, and 5P-phaAinternal, which produce PCR products of sizes 3.56 kb, 2.0 kb and 1.5 kb, respectively. These primers anneal at different locations within the gene construct (see FIG. 1A). Results revealed specific PCR size products in the transgenic lines (FIG. 2A lanes 4-6), confirming the presence of the phaA gene.

The DNA from T₀ and T₁ generation transgenic lines as well as from wild type plant (wt) was extracted and used for Southern-blot analysis (FIG. 2B-E). The flanking sequence probe of size 0.8 kb (FIG. 1B), which hybridizes with the trnI and trnA genes, allows detection of site-specific integration of the transgene cassette as well as the achievement of homoplasmy of the transgenic chloroplast genome. Additionally, the phaA probe (˜1.2 kb) was used to confirm the presence of the phaA gene. DNA from untransformed plants and transgenic lines were digested with BamHI (FIG. 1 A and B) and probed with either a flanking sequence probe or a phaA probe, resulting in the detection of a 10 kb transgenic chloroplast fragment (FIG. 2B-E, lanes 2-4). The detection of a 7.1 kb fragment by the flanking sequence probe in the wild type indicated that these chloroplasts lacked the integration of the foreign genes (FIGS. 2B and D, lane 1). The fact that no 7.1 kb size fragment (wt size) in the transgenic sample was observed indicated that homoplasmy had been achieved through the selection process even in T₀ (FIG. 2B, lanes 2-4) and was maintained in T₁ generation (FIG. 2D, lanes 2-4), confirming stable integration of foreign genes within all chloroplast genomes (to the limits of detection by Southern blots). The absence of any hybridizing fragment in the wild type when screened with the phaA probe indicated the absence of phaA gene (FIGS. 2 C and E, lane 1). If any unexpected size fragment were observed in the transgenic samples when probed with the transgene after prolonged exposure of Southern blots to x-ray film, nonspecific integration into the nuclear or mitochondrial genomes would be indicated. Such nonhomologous recombination was not observed.

Transcript abundance and stability from chloroplast transgenic lines were studied by northern-blot analysis using the gene specific probes phaA and aadA on total plant RNA (FIGS. 2 F and G). The chloroplast transgenic lines were expected to transcribe a 2,255 nt dicistron (aadA-phaA, FIG. 2, F and G) from the upstream 16S promoter (Prrn), in addition to a 1,384 nt monocistron (phaA) transcribed from the psbA promoter located upstream from the phaA gene (FIG. 2F, lanes 2-3). As expected, the monocistron was not detected with the aadA probe, showing that polycistrons are transcribed from the engineered Prrn promoter; less abundant polycistrons transcribed from the native 16S promoter were also detected (FIG. 2G). These results showed that both the monocistron and dicistron transcripts were abundant in the transgenic plants, because of the efficiency of the psbA and Prrn promoters, which are strong endogenous promoters. Additionally, larger size transcripts were detected, one of them correlating with the size of a transcript starting at the chloroplast native 16S promoter (Prrn) and terminating at the 3′ UTR psbA; the predicted size of this transcript is 4,723 nt. Other transcripts detected may be either read through (because 3′ UTR does not terminate transcription efficiently in chloroplast) or processed products.

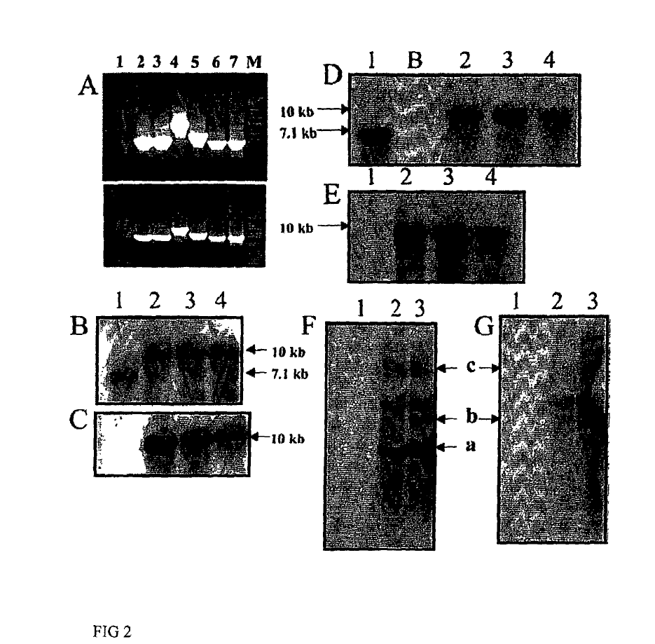

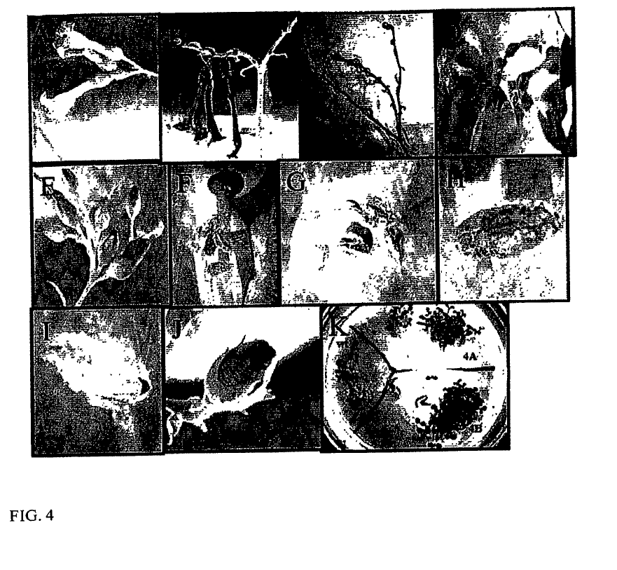

To confirm expression of β-ketothiolase in the chloroplast transgenic lines, untransformed and transformed plants were subjected to western-blot analysis by using anti-β-ketothiolase antibody. Chloroplast-synthesized β-ketothiolase treated with βP-mercaptoethanol (BME) and boiled, appeared mostly as monomeric forms (40.8 kDa), or in polymeric forms, which included the homotetrameric form (163 kDa, FIG. 3B, lanes 2-6). The homotetramer is the functional form of β-ketothiolase. No β-ketothiolase was detected in wild type samples (FIG. 3B lane 1). The appearance of a distinct band at 40.8 kDa in the Coomassie-stained sodium dodecyl sulfate-polyacrylamide electrophoresis (SDS-PAGE) gel (FIG. 3A, lanes 1-4) but not in the untransformed sample (lane 5) suggests that the chloroplast transgenic plants were expressing high levels of β-ketothiolase.

The activity of the chloroplast-expressed β-ketothiolase was measured in the thiolysis direction (breaking down acetoacetyl-CoA to acetyl-CoA) spectrophotometrically at 304 nm. The chloroplast transgenic lines showed β-ketothiolase activities that were up to 30-fold higher than previous levels demonstrated from nuclear transgenic plants. No endogenous β-ketothiolase activity was detected in untransformed tobacco plants (less than 0.0001 unit/mg plant protein; Table 1). The chloroplast transgenic lines showed levels of activity that reached 14.08 to 14.71 during normal photoperiod (16 hligh/8 hr dark, Table I). After 5 d of continuous illumination, the enzyme activity slightly increased in both transgenic lines (Table I). Thus, β-ketothiolase activity remained unchanged from light/dark photoperiod to continuous illumination, even though the phaA gene is under the control of strong psbA regulatory elements that should enhance translation in the light. The high levels of enzymatic activity correlated well with the high amounts of protein detected by the Coomassie-stained gel and western-blot analyses performed on total plant samples; these results suggested that the enzyme was in its biosynthetically active form (homotetramer). No adverse effects, such as growth reduction and chlorosis were observed in the transgenic lines hyperexpressing β-ketothiolase. Compartmentalization of proteins in chloroplasts has been shown to avoid pleiotropic effects, as previously reported for CTB (26), trehalose (24) and xylanase (25).

TABLE 1
β-Ketothiolase activity as a function of illumination

| | Illumination | Enzyme Activity |
|---|---|---|
| | Period Light/Dark | Units |
| Plant Line | h | μmol/min |
| Untransformed | 16 h/8 h | <0.0001 |
| 4A | 16 h/8 h | 0.215 |
| 4B | 16 h/8 h | 0.259 |
| Untransformed | 5 d of light | <0.0001 |
| 4A | 5 d of light | 0.247 |
| 4B | 5 d of light | 0.239 |
| Purified bacterial | na | 0.017 |
| β-ketothiolase | | |

Enzyme activity for transgenic lines 4A and 4B and untransformed tobacco plants at the respective illumination periods are shown. Protein samples were obtained by grinding 1 g of leaf tissue in liquid nitrogen, followed by the addition of β-ketothiolase extraction buffer (100 mM Tris-HCl, pH 8.1, 50 mM MgCl₂, 5 mM BME) and homogenization. Total plant protein concentration was determined by Bradford assay. Ten microliters of crude plant extract was used per assay. β-Keothiolase activity was measured spectrophotometrically at 304 nm for 60 s in the thiolysis direction.
na, Not applicable.

### EXAMPLE 12

### Characterization of Male Sterility

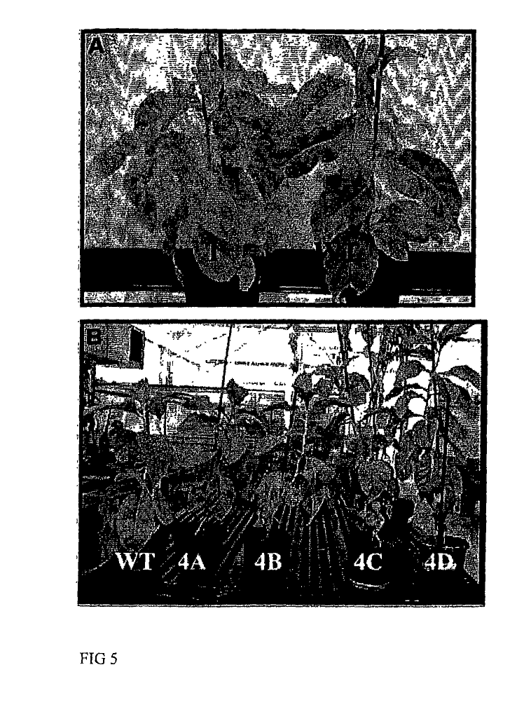

From the 10 T₀ transgenic lines expressing β-ketothiolase, 100% of the flowers produced by transgenic plants failed to develop fruit capsules and seeds, finally senescing and falling off (FIG. 4. A-C). The male sterility phenotype was maintained in T₁ generation transgenic lines. To (5A) and T₁ (FIG. 5B) generation transgenic plants showed no difference in growth and development when compared to untransformed tobacco plants under the same growth conditions. Like the parental line, the T₁ transgenic lines were not affected by hyper-expression of phaA and were phenotypically indistinguishable from untransformed control plants (FIG. 5B). Chlorophyll content analysis of three independent T₁ transgenic lines showed average chlorophyll content of 1.90±0.12 mg/g fresh weight. The chlorophyll content of three wild type plants average, 1.92±0.20 mg/g fresh weight. These results showed that the chlorophyll levels in the leaves of the transgenic lines expressing β-ketothiolase were similar to the levels in wild type tobacco plants, confirming that chloroplast biosynthetic functions and integrity of the thylakoide membranes, although β-ketothiolase was hyperexpressed.

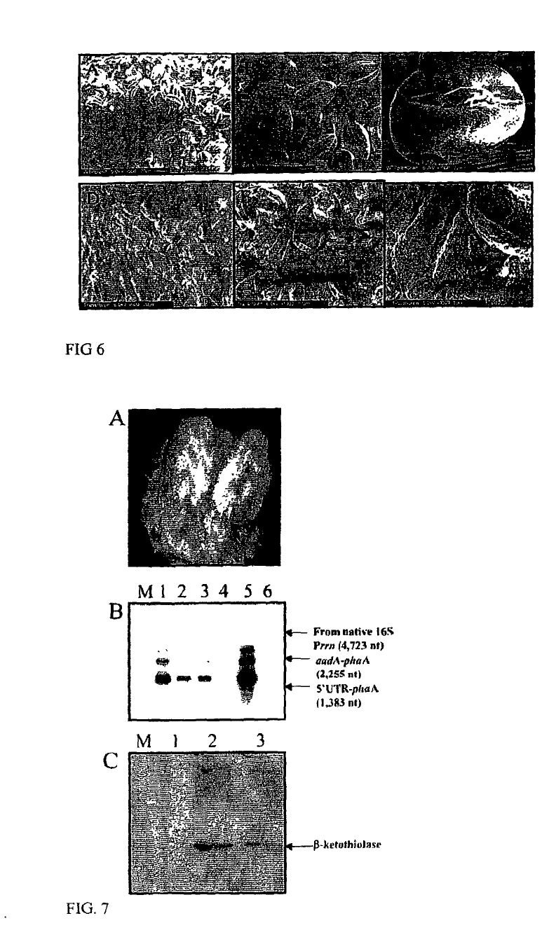

However, the chloroplast transgenic lines showed specific defects in anther development and failed to produce viable pollen. The anthers were characterized by the lack of pollen grains (FIG. 4H) or when pollen grains were formed, they were abnormal with a collapsed phenotype (FIG. 6). Additionally, the stamens were shorter (FIG. 4F) than in wild type plants (FIG. 4G), adding a degree of severity to the male sterile phenotype. To investigate that plants were sterile due to lack of pollen or non-viable pollen, a total of 15 emasculated wild type flowers from three untransformed plants (five flowers from each untransformed plant) were pollinated with pollen collected from anthers from transgenic lines 4A, 4B, and 4C. Pollen from each transgenic line was used to pollinate a total of five untransformed emasculated flowers. All of these crosses failed to produce seeds. The shorter stamens may be a consequence of the failure of the cells to elongate in the central and upper parts of the anther filament. To investigate the possibility of female infertility affecting the transgenic plants, they were fertilized with pollen from wild type. The transgenic plants developed normal fruit capsules (FIG. 4J) and normal seeds that were able to germinate and develop normally (FIG. 4K). The T₁ seedlings grew well in the medium supplemented with 500 μg/ml spectinomycin and were identical to the parental line (T₀) and Southern blot analysis showed the presence of the gene construct (FIG. 2, D and E).

Scanning electron microscopy was performed on transgenic anthers as well as wild type anthers to further characterize male sterility in transgenic plants. The SEM revealed that the pollen grains in the transgenic anthers exhibited collapsed morphology and consisted of a heterogeneous population with respect to size and shape (FIG. 6, D-F). Wild type anthers showed a homogenous population of pollen grains of uniform size and shape (FIG. 6, A-C). The apparent lack of turgidity of the transgenic pollen may be produced by lack of intracellular material, resulting in the distorted and collapsed morphology. The aberrant pollen morphology observed under the SEM may account for the inability of the transgenic plant to produce seeds.

### EXAMPLE 13

### β-Ketothiolase Expression in Anthers

Plastids in anthers may be in low abundance when compared to the numbers in leaves, but they produce enough β-ketothiolase to affect pollen development in anthers. As shown in FIG. 7A, flowers and specifically anthers were green during anther development (stage 1 of flower development is shown). This means that chloroplasts are present and are metabolically active. Northern-blot analysis of leaves showed that the mRNAs coding for β-ketothiolase are found in monocistronic and polycistronic form, the monocistron form being the most abundant transcript (FIG. 2F). The same pattern was maintained in the phaA transcripts in flowers and anthers (FIG. 7B). Northern-blot analysis of transgenic flowers and anthers showed that transcription of the phaA gene occurs in the flower and anther; this was expected because Prrn and psbA promoters are constitutive promoters, allowing transcription to occur in both photosynthetic as well as non-photosynthetically active plastids (FIG. 7B). The translation of the phaA monocistron is under the light regulated psbA 5′ regulatory sequence but because the flowers, as well as anthers are green, containing photosynthetically active chloroplasts, translation was quite efficient (FIG. 7C). β-ketothiolase was detected by western-blot in both whole flowers and anthers from transgenic plants, confirming that the β-ketothiolase was present during anther development, and should play a role in the male sterility phenotype (FIG. 7C).

### EXAMPLE 14

### Anther Development and Male Sterility

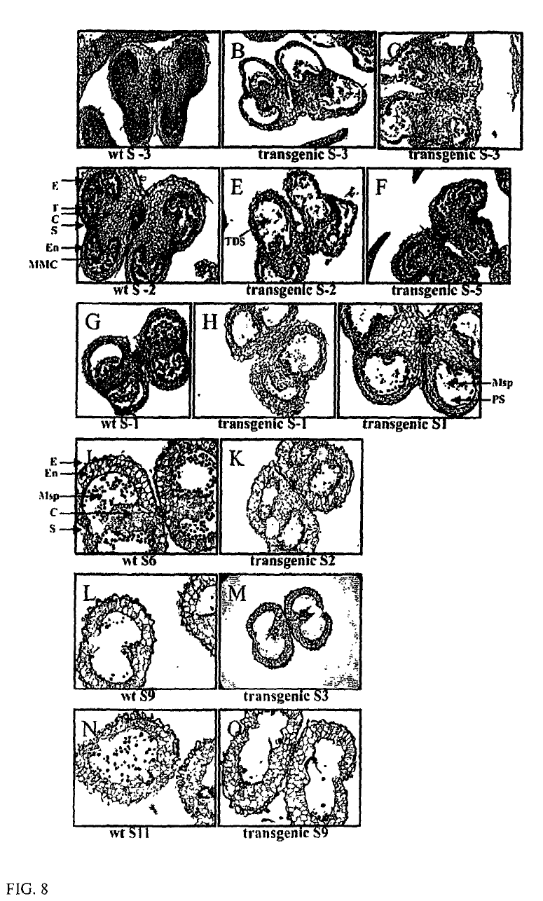

Analysis of anther development revealed that the anthers of the transgenic lines followed an accelerated pace in their development and maturation resulting in aberrant tissue patterns (FIG. 8). At stage −3 of flower development the WT plants showed a normal pattern of tissue development, where all major tissues were differentiated, the anther had acquired its characteristic shape, all tissues were interconnected and there was presence of callose depositions between microspore mother cells. This pattern was not followed in the transgenic lines, which at stage −3, showed characteristics of a more advanced stage. The transgenic anthers at stage −3, showed the microspores in tetrads, with stomium differentiation occurring and the tapetum was shrunken and broken. This pattern presented characteristics of more advanced stages of flower development, which included stages +1 and +2. Additionally, the transgenic anthers showed abnormal development of the epidermis and endothecium, probably resulting in the aberrant shape of anthers. The anthers of the transgenic lines at stage −2 were also advanced in the aberrant phenotype with the microspores already separated, a developmental step that should have been observed at stage +2. Again the tapetal layer was broken. We noticed that the transgenic anthers at stage −5, which is a very early stage of flower development, showed great similarities with stage −2 in the wild type plants, but aberrant pattern of tissue development could still be observed. At stage −1 in transgenic line the tapetum was shrunken and discontinuous and formation of pollen grains was evident at stage 1. These morphological changes observed in stage −1 and +1 should be observed at much later stages, +3 and +4. At early stages of floral development (stages −5 to +1), transgenic lines showed accelerated anther development, which averaged +3 stages ahead from the wild type plants. At late stages of floral development, accelerated phenotype increased even more, at an average of 4 to 6 stages ahead of wild type. At stage +2 in the transgenic lines, cells adjacent to the stomium had degenerated and only remnants of the tapetum were observed. The thickening of the outer wall is accompanied by enlarged endothecium and vacuolation, which greatly decreased the inner space of the locules, crushing pollen grains and resulted in the irregular shape and collapsed phenotype. The developmental changes observed in the transgenic anthers at stage +2, although aberrant, were similar to the ones observed in wt at stage +6. In the wild type, abundant normal pollen grains were observed. Almost complete degradation of the connective tissue that separates the pollen sac occurred at stage +3 in transgenic anthers, while this occurred at stage +9 in wild type plants. Finally, both wild type and transgenic anthers were bilocular, connective tissue was absent but the major difference was that abundant pollen was present in the WT (stage +11) but not in transgenic anthers (transgenic, stage +9). Additionally, the pollen grains formed in transgenic anthers were collapsed. The data presented here allows us to understand the effect of β-ketothiolase during anther development in the transgenic lines.

Anther development is a very complex process involving the coordination of several genes and the specific development and maturation of several tissues and cells (13); any defect in these well-coordinated processes may lead to dysfunctional pollen. Many male-sterility systems produced by mutations or nuclear expressions of foreign proteins have shown to interfere with the function or differentiation of tapeturn, indicating that this tissue is essential for the production of viable pollen (17). Here we observed that the tapetum of the transgenic lines was severely impaired. The tapetum is critical for the development of pollen by secreting essential substances such as proteins (13), carbohydrates (17) and lipids (14) into the locules. Developing microspores and the surrounding tapetal cells have been shown to be particularly active in lipid metabolism (14). The precise differentiation and maturation of tapetum with respect to microspore development is of major importance for the successful production of pollen. Here we observed complete dysfunction in the anther tissue differentiation patterns, which may be caused by an alteration in chloroplast fatty acid metabolism in the transgenic lines expressing the phaA gene, affecting the development of pollen grains.

### EXAMPLE 15

### Reversibility of Male Fertility

To test whether depletion of the acetyl-CoA pool destined for de novo fatty acid biosynthesis in chloroplast by β-ketothiolase is the cause of the male sterility phenotype, the inventors explored whether continuous illumination could revert male fertility of the chloroplast transgenic lines. The photoperiod experiments represent an indirect test of the proposed basis for male sterility in this system. Acetyl-CoA carboxylases (ACCase) carries the first committed step in fatty acid biosynthesis, which involves the conversion of acetyl-CoA to malonyl-CoA. Because acetyl-CoA is not imported into plastids from the cytoplasm, it should be synthesized in this organelle; the expression of phaA in the chloroplast should increase the competition for the same pool. Therefore, if β-ketothiolase and ACCases are competing for the same acetyl-CoA pool, β-ketothiolase will outcompete ACCases when it is hyperexpressed in transgenic chloroplasts; this could decrease the supply of acetylCoA for fatty acid biosynthesis. Therefore, conditions that could divert the acetyl-CoA pool back to the fatty acid biosynthesis pathway might restore the fatty acid biosynthesis. It has been shown that the intermediates of fatty acid biosynthesis change during the transition to darkness in leaves and chloroplasts in a manner consistent with control at the levels of ACCase (37). Recent reports have shown light-dependent regulation of ACCase by the redox status of the plastid whereby the enzyme is more active under the reducing conditions observed in light (38, 39).

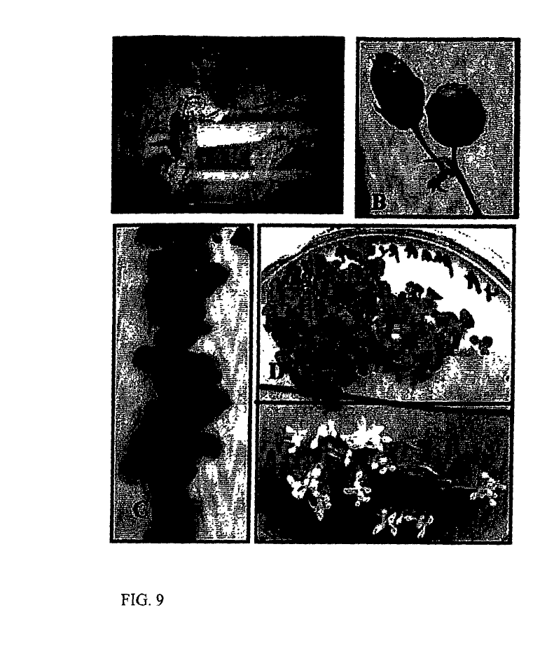

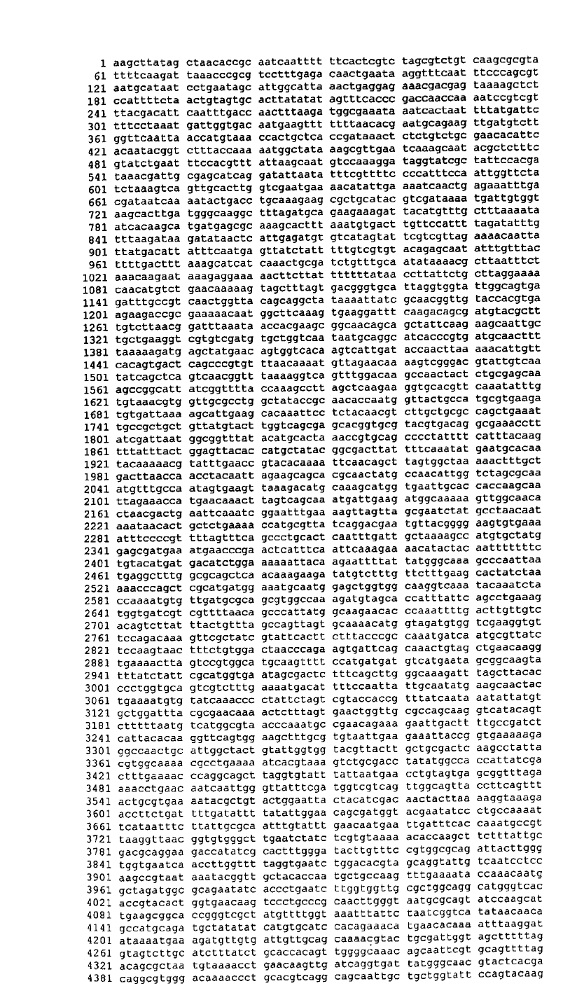

To test the inventors' hypothesis, two independent transgenic lines 4A and 4B were exposed to continuous light for a period of 10 days. These plants had been previously characterized and shown to be 100% male sterile unable to produce any fruit capsules or seeds. These plants were isolated from all other plants (transgenic and wild types) in a growth chamber at the first indication of flower bud development (before any flower was opened), and the flowers were covered with transparent plastic bags to avoid cross-pollination. We observed that from a total of 20 flowers produced during the 10 days of illumination by the two transgenic plants, 4 flowers were able to produce pollen (FIG. 9A), normal length anther filaments (FIG. 9A), developed fruit capsules (FIG. 9B) and produced seeds (FIG. 9C). Transgenic line 4A produced 3 viable flowers during the 10 day continuous light photoperiod, all of them being produced between days 8 and 10. Transgenic line 4B produced one viable flower at day 9 and was able to produce additional viable flowers three days after the 10 days assay was completed. The seeds recovered (FIG. 9C) from the reverted male fertility study were able to genninate in a medium with 500 μg/mL spectinomycin (FIG. 9D), indicating that these seedlings were transgenic and contained the transgene; wild type tobacco seedlings were bleached (FIG. 9E). These findings support the hypothesis that an increase in ACCase activity outcompetes, at least partially, the removal of acetyl-CoA by β-ketothiolase. Additionally, this line of evidence shows that male sterility is caused by the effect of β-kethotiolase expression in the chloroplast and not by any other unknown process

### SUMMARY OF CONCLUSIONS REACHED FROM EXAMPLES 1-15

The inventors have shown that the hyperexpression of β-ketothiolase via chloroplast transformation results in normal growth and pigmentation, even when the activity of the enzyme was very high. Successful expression of β-ketothiolase in transgenic plants showed that this enzyme could be safely expressed in the chloroplast and suggests that the complete PHB pathway needs to be expressed in order to cause the stunted phenotype. Although the inventors observed no growth reduction in the chloroplast transgenic lines expressing β-ketothiolase, 100% male sterility was observed. This is a significant advancement in the art because of the importance of the production of male sterile lines in gene containment and hybrid seed production (11). Because the expression of β-ketothiolase did not disrupt growth and normal development, with the exception of the lack of pollen formation, the expression of β-ketothiolase may be used as a mechanism to generate a male sterility system, producing 100% infertility that can be applied to different plant species. Concerns related to constitutive expression of phaA are easily overcome by restricting phaA expression to the anthers, where pollen formation occurs. Such transgenic plant systems expressing a chloroplast targeted T7 RNA polymerase via the nuclear genome, regulating the expression of a chloroplast integrated transgene under the g10 T7 promoter has been reported in the literature (40, 41). A similar approach in which the T7 RNA polymerase gene regulated by an anther specific promoter may be used to specifically induce phaA expression in anther plastids for transgene containment or hybrid seed production.

The inventors have demonstrated that it is possible to revert the chloroplast transgenic lines to fertility by continuous light exposure. Not to be bound by any particular theory, this supports a mechanism of action in which β-ketothiolase depleted the pool of acetyl-CoA in the chloroplast, but by increasing acetyl-CoA carboxylase (ACCase) activity by continuous light (37, 38, 39), ACCase was able to compete more effectively for acetyl-CoA, thereby increasing the levels of plastidic fatty acid biosynthesis. Developing microspores and surrounding tapetal cells have been shown to be particularly active in lipid metabolism (13), which is especially needed for the formation of the exine pollen wall from sporopollenin (42, 43). Support for the normal growth and development observed in the chloroplast transgenic lines expressing phaA also comes from studies where a mutant plant with 10% activity of acyl-CoA synthase showed normal content of lipids in leaves and normal growth (44), indicating that under normal growth conditions, even severely impaired plants in fatty acid biosynthesis were able to grow normally. Chloroplasts genetic engineering approach offers a number of attractive advantages, including high-level transgene expression (45), multi-gene engineering in a single transformation event (45-48), transgene containment via maternal inheritance (49, 50), lack of gene silencing (24, 31, 45), position effect due to site specific transgene integration (51) and lack of pleiotropic effects due to sub-cellular compartmentalization of transgene products (24-26). Genetically engineered cytoplasmic male sterile via the chloroplast genome may be used for the safe integration of foreign genes via the nuclear genome and in those rare cases where plastids genomes are paternally or biparentally transmitted (50). Recently, plastid transformation was demonstrated in carrot (52), showing hyperexpression of the transgene in non-green plastids to levels of up to 75% the expression in leaf chloroplast. Additionally, plastid transformation of recalcitrant crops such as cotton (53) and soybean (54) allows the application of the cytoplasmic male sterile system to commercially important crops.

An important consideration for hybrid development using CMS systems is the requirement, or not, for male-fertility restoration. For vegetable, fruit, or forage crops, restoration of male fertility in the hybrid is not necessary. This simplifies the production of hybrids because effort can concentrate on maintaining line development, without concern whether the pollinator restores male fertility in the hybrid. For crops with seeds as the economically important product, such as canola, sunflower, or maize, one or both of the hybrid's parents must bring in male-fertility restoration factors or the male-sterile hybrid seed must be blended with male-fertile hybrid seed (4). In currently available cytoplasmic male sterile lines, nuclear genome controls various restoration factors and such factors are often located at multiple loci and are poorly understood. However, the inventors show that restoration of male fertility may be achieved by changing conditions of illumination. Also, in the case of β-ketothiolase induced male sterility, fertility can be obtained by the use of regulatory or inducible elements instead of constitutive expression. Thus, this is a novel approach for creating male sterile transgenic plants, which may help advance the field of plant biotechnology through effective transgene containment.

All patents, patent applications, publications, texts and references discussed or cited herein are incorporated by reference to the same extent as if each individual publication or patent application was specifically and individually set forth in its entirety. Nothing herein is to be construed as an admission that the invention is not entitled to antedate such disclosures by virtue of prior invention. In addition, all terms not specifically defined are first taken to have the meaning given through usage in this disclosure, and if no such meaning is inferable, their normal meaning. Where a limitation is described but not given a specific term, a term corresponding to such limitation may be taken from any references, patents, applications, and other documents cited herein, or, for an application claiming priority to this application, additionally from an Invention Disclosure Statement, Examiner's Summary of Cited References, or a paper otherwise entered into the file history of this application.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. Thus, for the above variations and in other regards, it should be understood that the examples and embodiments described herein are for illustrative purposes only and that various modifications or changes in light thereof will be suggested to persons skilled in the art and are to be included within the spirit and purview of this application and the scope of the appended claims.

### REFERENCES

1. Bateson, W., & Gairdner, A. E. (1921) J. Genet. 11, 269-275.
2. Chittenden, R. J., & Pellow, C. A. (1927) Nature 119, 10-12.
3. Jones, H., & Clarke, A. (1943) Proc. Amer. Soc. Hort. Sci. 43, 189-194.
4. Havey, M. J. (2004) In Molecular Biology and Biotechnology of Plant Organelles, eds. Daniell, H. & Chase, C. D. (Kluwer Academic Publishers, The Netherlands), pp 617-628.
5. Pring, D., & Lonsdale, D. M. (1989) Ann. Rev. Phytopathol. 27, 483-502.
6. Frederickson, D. E., Mantle, P. G., & de Milliano, W. A. J. (1994) Plant Pathol. 43, 27-32.
7. Bandyopadhyay, R., Frederickson, D. E., McLaren, N. W., Odvody, G. N., & Ryley, M. J. (1998) Plant Dis. 82, 356-367.
8. Smith, B., & Crowther, T. (1995) Euphytic 86, 87-94.
9. Marian, i C., De Beuckeleer, M., Truettner, J., Leemans, J.,& Goldberg, R. B. (1990) Nature 347, 737-741.
10. Hernould, M., Suharsono, S., Litvak, S., Araya, A., & Mouras, A. (1993) Proc. Natl. Acad. Sci. USA 90, 2370-2374.
11. Perez-Prat, E., & van Lookeren Campagne, M. M. (2002) TRENDS Plant Sci. 7, 199-203.
12. Kriete, G., Niehaus, K., Perlick, A. M., Puhler, A., & Broer, I. (1996) Plant J. 9, 809-818.
13. Yui, R., Iketani, S., Mikami, T., & Kubo, T. (2003) Plant J. 34, 57-66.
14. Zheng, Z., Xia, Q., Dauk, M., Shen, W., & Selvaraj, G., Zou, J. (2003) Plant Cell 15, 1872-1887.
15. Hemould, M., Suharsono, Zabaleta, E., Carde, J. P., Litvak, S., Araya, A., & Mouras, A. (1998) Plant Mol. Biol. 36, 499-508.
16. Napoli, C. A., Fahy, D., Wang, H. Y., & Taylor, L. P. (1999) Plant Physiol. 120, 615-622.
17. Goetz, M., Godt, D. E., Guivarc'h, A., Kahrnann, U., Chriquli, D., & Roitsch, T. (2001) Proc. NatI. Acad, Sci. USA 98, 6522-6527.
18. Stewart, C. N. Jr., Halfhill, M. D., & Warwick, S. I. (2003) Nat. Rev. Genet. 4, 806-817.
19. Poirier, Y., Dennis, D. E., Klomparens, K., & Somerville. C. (1992) Science 256, 520-523.
20. Nawrath, C., Porier, Y., & Somerville, C. (1994) Proc. Natl Acad. Sci. USA 91, 12760-12764.
21. Bohmert, K., Balbo, I., Kopka, J., Mittendorf, V., Nawrath, C., Poirier, Y., Tischendorf, G., Trethewey, R. N., & Willmitzer, L. (2000) Planta 211, 841-845.
22. Lossl, A., Eibl, C., Harloff, H. J., Jung, C., & Koop H. U. (2003) Plant Cell Rep. 21, 891-899.
23. Bohmert, K., Balbo, I., Steinbuchel, A., Tischendorf, G., & Willmitzer, L. (2002) Plant Physiol. 128, 1282-1290.
24. Lee, S. B., Kwon, H. B., Kwon, S. J., Park, S. C., Jeong, M. J., Han, S. E., Byun, M. O., & Daniell, H. (2003) Mol. Breed. 11, 1-13.
25. Leelavathi, S., Gupta, N., Maiti, S., Ghosh, A., & Reddy, V. S. (2003) Mol. Breed. 11, 59-67.
26. Daniell, H., Lee, S. B., Panchal, T.,& Weibe, P. O. (2001a) J. Mol. Biol. 311, 1001-1009.
27. Daniell, H., Datta, R., Varma, S., Gray, S., & Lee, S. B. (1998). Nature Biotechnol. 16, 345-348.
28. Eibl, C., Zou, Z., Beck, A., Kim, M., Mullet, J., & Koop, H. U. (1999): Plant J. 19, 333-345.
29. Fernandez-San millan, A., Mingo-Castel, A., Miller, M., & Daniell, H. (2003) Plant Biotechnol. J. 1, 71-79.
30. Daniell, H., Cohill, P. R., Kumar, S., Dufoummantel, N., & Dubald, M. (2004c). In Molecular Biology and Biotechnology of Plant Organelles, eds. Daniell, H., & Chase, C. (Kluwer Acaderic Publishers, The Netherlands), pp. 437-484.
31. Dhingra, A., Portis, A. R. Jr., & Daniell, H. (2004) Proc. Natl. Acad. Sci. USA 101, 6315-6320.
32. Marchfelder, A., & Binder, S. (2004). In Molecular Biology and Biotechnology of Plant Organelles, eds. Daniell, H., & Chase, C. (Kluwer Academic Publisher, The Netherlands), pp. 257-290.
33. Daniell, H. (1997) Methods Mol. Biol. 62, 453-488.
34. Daniell, H., Ruiz, O. N., & Dhingra, A. (2004a). Chloroplast genetic engineering to improve agronornic traits. Methods Mol. BioL 286, 111-137.
35. Kumar, S., & Daniell, H. (2004) Methods Mol Biol. 267, 365-383.
36. Daniell, H., Muthukumar, B., & Lee, S. B. (2001b) Curr. Genet. 39, 109-116.
37. Post-Beittenmiller, D., Jaworski, J. G., & Ohlrogge, J. B. (1991) J Biol. Chem. 266, 1858-1865.
38. Page, R. A., Okada, S., & Harwood, J. L. (1994) Biochim. Biophys. Acta 1210, 369-372.
39. Kozaki, A., Kamada, K., Nagano, Y., Iguchi, H., & Sasaki, Y. (2000) J. Biol. Chem. 275, 10702-10708.
40. McBride, K. E., Schaaf, D. J., Daley, M., & Stalker, D. M. (1994) Proc Natl Acad Sci USA. 91,
41. Magee, A. M., & Kavanagh, T. A. (2002) J Experimental Botany 53, 2341-2349.
42. Wang, A., Xia, Q., Xie, W., Dumonceaux, T., Zou, J., Datla, R., Selvaraj, G. (2002) Plant J. 30, 613-623.
43. Ariizumi, T., Hatakeyama, K., Hinata, K., Lnatsugi, R., Nishida, I., Sato, S., Kato, T., Tabata, S., & Toriyama, K. (2004) Plant J. 39, 170-81.
44. Schnurr, J. A., Shockey, J. M., de Boer, G. J., & Browse, J. A. (2002) Plant Physiol. 129, 1700-1709.
45. De Cosa, B., Moar, W., Lee, S. B., Miller, M., & Daniell, H. (2001) Nat. Biotechnol. 19, 71-74.
46. Daniell, H., & Dhingra, A. (2002a) Curr. Opin. Biotechnol. 13, 136-141
47. Ruiz, O. N., Hussein, H., Terry, N., & Daniell, H. (2003) Plant Physiol. 132, 1344-1352.
48. Lossl, A., Eibl, C., Harloff, H. J., Jung, C., & Koop, H. U. (2003) Plant Cell Rep. 21, 891-899.
51. Daniell, H. (2002) Nat. Biotechnol. 20, 581-586.
52. Hagemann, R. (2004) In Molecular Biology and Biotechnology of Plant Organelles, eds. Daniell, H., & Chase, C. (Kluwer Academic Publisher, The Netherlands), in press.
53. Daniell, H., Khan, M. S., & Allison, L. (2002b) Trends Plant Sci. 7, 84-91.
54. Kumar, S., Dhingra, A., & Daniell, H. (2004a). Plant Physiol. in press.
55. Kumar, S., Dhingra, A., & Daniell, H. (2004b). Plant Mol. Biol. in press.
56. Dufourmantel, N., Pelissier, B., Garçon, F., Peltier, J. M., & Tissot, G. (2004) Plant. Mol. Biol. in press.
57. Senior, P. J., & Dawes, E. A. (1973) Biochem. J. 134, 225-238.
58. Koltunow, A. M., Truettner, J., Cox, K. H., Wallroth, M., & Goldberg, R. B. (1990) Plant Cell. 2,
   U.S. Pat. Nos. 6,680,426 and 6,642,053; PCT publication WO 04/005480, WO 99/10513 and U.S. patent publication 2002/0174453 are also incorporated herein by reference for purposes of providing basic chloroplast transformation and plant regeneration principles.

What is claimed is:

1. A stable plastid transformation and expression vector which comprises an expression cassette comprising, as operably linked components in the 5′ to the 3′ direction of translation, a promoter operative in said plastid, a selectable marker sequence, a heterologous polynucleotide sequence coding for β-ketothiolase activity and comprising at least 70% identity to a phaA gene, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

2. A vector of claim 1, wherein the plastid is selected from the group consisting of chloroplasts, chromoplasts, amyloplasts, proplastide, leucoplasts and etioplasts.

3. A vector of claim 1, wherein the selectable marker sequence is an antibiotic-free selectable marker.

4. A vector of claim 1 competent for stably transforming a plastid genome of different plant species wherein the flanking DNA sequences are homologous to a transcriptionally active spacer sequence of the target plastid genome.

5. A stably transformed plant which comprises plastid stably transformed with the vector of claim 1 or the progeny thereof, including seeds.

6. A stably transformed plant of claim 5 which is a monocotyledonous or dicotyledonous plant.

7. A stably transformed plant of claim 6 which is maize, rice, grass, rye, barley, oat, wheat, soybean, peanut, grape, potato, sweet potato, pea, canola, tobacco, tomato or cotton.

8. A stably transformed plant of claim 5 which is edible for mammals and humans.

9. A stably transformed plant of claim 5 in which all the chloroplasts are uniformly transformed.

10. A stably transformed plant of claim 5 in which the transformed plastid of the plants including subsequent generations are capable of enhanced levels of expression.

11. A method for obtaining a stably transformed plant comprising male sterility, said method comprising introducing an integration and expression vector of claim 1 into a plastid genome of plant tissue or cells to produce stably transformed tissue or cells, and regenerating a plant from said stably transformed tissue or cells.

12. A method for introducing a polynucleotide sequence encoding a β-ketothiolase gene into a plastid, said method comprising: introducing a plant cell with a plastid expression vector adsorbed to a microprojectile, said plastid expression vector comprising as operably linked components, a polynucleotide sequence containing at least one plastid replication origin functional in a plant plastid, a transcriptional initiation region functional in said plant plastid, at least one heterologous polynucleotide sequence encoding Acinetobacter sp. β-ketothiolase gene, and a transcriptional termination region functional in said cells, whereby said heterologous polynucleotide sequence is introduced into plastid in said plant cell.

13. The stably transformed plant according to claim 5, wherein expression of β-ketothiolase is regulated so as to occur only in anther tissue.

14. The stably transformed plant according to claim 5, wherein said male sterility is reversible.

15. The stably transformed plant of claim 14, wherein said male sterility is reversed by changing illumination conditions subjected to said plant.

16. The vector of claim 1, wherein said promoter is an anther-specific promoter.

17. A stably transformed transcription/translation active chloroplast genome of a target plant, which chloroplast genome has been transformed with an expression cassette which comprises a protein having β-ketothiolase activity and is encoded by a heterologous DNA sequence comprising at least 70 percent identity to a phaA gene, regulated by control sequences to provide expression of the heterologous DNA sequence in the chloroplast genome of the target plant, and plant DNA flanking each side of the expression cassette which facilitated stable integration of the DNA into the target chloroplast genome by homologous recombination, which DNA is inherited through organelle replication in daughter cells, wherein the expression cassette is inserted into a transcriptionally active spacer region between two genes located on the same DNA strand of the target higher plant's chloroplast genome.
